(19) United States
(12) Patent Application Publication
(10) Pub. No.: US 2016/0089383 A1
(43) Pub. Date: Mar. 31, 2016

(54) NUTRITIONAL SUPPLEMENTS

(71) Applicant: EVERBLOOM, LLC, Miami Beach, FL (US)

(72) Inventors: Paola Macri, Jersey City, NJ (US); Leyda E. Bowes, Coral Gables, FL (US); Jeffrey L. Kosiba, Milton, MA (US)

(21) Appl. No.: 14/868,670

(22) Filed: Sep. 29, 2015

Related U.S. Application Data
(60) Provisional application No. 62/056,663, filed on Sep. 29, 2014.

(51) Int. Cl. A61K 31/593; A61K 31/353; A23L 1/00; A61K 9/48; A23L 1/303; A23L 1/30; A61K 31/592; A61K 31/047

(52) U.S. Cl. CPC A61K 31/593; A61K 31/592; A61K 31/353; A61K 31/047; A61K 9/4833; A23L 1/303; A23L 1/30; A23L 1/0029; A23V 2002/00

(57) ABSTRACT

Provided are nutritional supplement preparations and methods for enhancing the outcome of a non-invasive tissue remodeling procedure, for providing the nutritional supplement to a subject, and for preparing such supplements.

### CROSS-REFERENCE TO RELATED APPLICATIONS

This application claims the benefit of U.S. Provisional Application No. 62/056,663, filed on Sep. 29, 2014, entitled “Nutritional Supplements,” which is hereby expressly incorporated by reference into the present application.

### FIELD OF THE INVENTION

The present application relates generally to nutritional supplements for supporting the body's response to non-invasive tissue remodeling procedures.

### BACKGROUND

“Subcutaneous tissue” includes tissue lying beneath the dermis and includes subcutaneous fat, or adipose tissue that may be composed primarily of lipid-rich cells, or adipocytes. Cellulite, or lumpy, bumpy, irregular “peau d'orange” or “cottage cheese”-like skin, forms when subcutaneous fat deposits into packets between bands of fibrous tissues that connect the dermis with the underlying tissues. Excess adipose tissue is thought to magnify the unattractive appearance of cellulite. The adipocyte has a large amount of cytoplasm that serves as a storage depot for excess triglycerides in the blood that are not utilized for energy. As adipocytes continue to enlarge within their intralobular and interlobular fascial compartments, they create “bulges” or convex distensions of soft tissue that then modify its contours.

Cellulite and excessive amounts of adipose tissue are often considered to be unappealing. Accordingly, a number of surgical and non-surgical procedures have been developed which remove such excess subcutaneous adipose tissue and cellulite.

Non-invasive body sculpting or tissue remodeling procedures have become popular, being safer, less traumatic and with quicker recovery, fewer side effects, and less discomfort relative to invasive or minimally invasive procedures such as liposuction, laser-assisted liposuction, and mesotherapy. Such non-invasive procedures include fat cooling methods (crylipolysis) (see, e.g., U.S. Pat. No. 7,367,341; U.S. Pub. No. 2005/0251120), suction and massage (U.S. Pub. No. 2008/0262574), and methods using radiofrequency energy (U.S. Pub. No. 2006/0036300), high frequency focused ultrasound (HIFU) radiation (see, e.g., U.S. Pat. Nos. 7,258,674 and 7,347,855), or low level light energy (U.S. Pat. No. 5,143,063) (see, e.g., Mulholland et al. (2011) Clin. Plastic Surg. 38:503-520). These procedures are thought to improve the look of fatty tissue by injuring adipocytes, the cells that store excess lipids. Damaged adipocytes may recover and heal or may eventually die if the procedure induces apoptosis, a process of programmed cell death, or if it negatively affects adipocyte size by decreasing lipogenesis (differentiation) or by stimulating lipolysis or adipocyte necrosis.

Despite being non-invasive, and hence causing less pain and a shorter recovery time, the results of these procedures take a longer time to fully develop. From one to six months is often the time quoted to see maximum improvement in the appearance of treated fatty tissue after the procedure. In addition, there have been side-effects noted, including paradoxical hyperplasia of the fatty tissue after cryolipolysis (Jalian et al. (2014) JAMA Dermatol. 150(3):317-9).

Thus, what are needed are methods and nutritional supplements which support and/or enhance the result of non-invasive tissue remodeling procedures while lessening the incidence of certain side effects.

### SUMMARY

It has been discovered that the synergistic combination of select concentrations of genistein, resveratrol, Vitamin D, and quercetin supports the body's natural responses to certain non-invasive injury. This discovery has been exploited to provide the present invention, which, in part, includes a nutritional supplement that supports the body's natural response to injury from a non-invasive tissue remodeling procedure, thereby resulting in an improvement of the appearance of treated fatty tissue.

In one aspect, the disclosure provides a nutritional supplement preparation comprising Vitamin D at 400 IU to 10,000 IU, genistein at 10 mg to 250 mg, resveratrol at 30 mg to 1,000 mg, and quercetin at 40 mg to 1,000 mg. In some embodiments, Vitamin D is Vitamin D2, Vitamin D3 (cholecalciferol), or a combination thereof. In certain embodiments, resveratrol comprises trans-resveratrol. In some embodiments, quercetin comprises quercetin dihydrate. In particular embodiments, the nutritional supplement preparation comprises cholecalciferol at 400 IU to 10,000 IU, genistein at 10 mg to 250 mg, trans-resveratrol at 30 mg to 1,000 mg, and quercetin dihydrate at 40 mg to 1,000 mg.

In some embodiments, the nutritional supplement preparation is in a unit dose form. In certain embodiments, this preparation contains cholecalciferol at 600 IU to 5,000 IU, genistein at 20 mg to 125 mg; trans-resveratrol at 50 mg to 600 mg, and quercetin dihydrate at 100 mg to about 500 mg. In another embodiment, the preparation contains cholecalciferol 1,000 IU to 5,000 IU, genistein at 17 mg to 100 mg, trans-resveratrol is present at 100 mg to 500 mg, and quercetin dihydrate at 100 mg to 200 mg.

In some embodiments, the supplement preparation further comprising a pharmaceutically-acceptable excipient, carrier, or diluent. In certain embodiments, the supplement preparation further comprises a filler, extender, excipient, binder; humectant, disintegrating agent, solution-retarding agent, wetting agent, absorbent, coloring agent, buffering agent, and/or combinations thereof.

In particular embodiments, the supplement preparation is in an orally available form, and in certain embodiments, this form is a capsule, pill, tablet, dragee, powder, gummy chew, or granule. In other embodiments, the supplement preparation is in emulsion, microemulsion, solution, suspension, syrup, or elixir form. In some embodiment, the supplement preparation is in time-release form.

The disclosure also provides a method of enhancing the outcome of a non-invasive tissue remodeling procedure, the outcome comprising an improved aesthetic appearance of a body area having undergone the procedure. This method comprises: examining the body area of the patient before the procedure; administering to the subject an amount of a nutritional supplement which supports the body's response to the procedure, and examining the body area after the procedure has been performed, the amount of nutritional supplement comprising: Vitamin D at 400 IU to 10,000 IU; genistein at 10 mg to 250 mg; resveratrol at 30 mg to 1,000 mg; and quercetin at 40 mg to 1,000 mg, wherein the treated body area has an improved aesthetic appearance relative to the appearance of the area before undergoing the procedure.

In some embodiments, the nutritional supplement administered comprises Vitamin D2, Vitamin D3, or a combination thereof. In some embodiments, the nutritional supplement administered comprises quercetin dihydrate. In certain embodiments, the nutritional supplement administered comprises trans-resveratrol. In particular embodiments, the nutritional supplement administers comprises: Vitamin D3 at 400 IU to 10,000 IU; genistein at 10 mg to 250 mg; trans-resveratrol at 30 mg to 1,000 mg; and quercetin dihydrate at 40 mg to 1,000 mg,

In some embodiments, the step of examining the body area of a patient that has undergone the procedure is performed multiple times of increasing length after the procedure, and the improved aesthetic appearance of the treated body area is detected in a shorter period time after the procedure relative to the period of time it takes to detect an improved aesthetic appearance of a body area of a patient who has undergone the procedure and who has not been administered the nutritional supplement. In certain embodiments, the examining steps comprise photographing the body area and/or taking caliper measurements of the area. In some embodiments, the patient is weighed the patient before and after having undergone the treatment.

In certain embodiments, the nutritional supplement is administered before the procedure, and in particular embodiments, the supplement is administered daily from 1 day to 60 days before the procedure. In other embodiments, the nutritional supplement being administered after the procedure, and in particular embodiments, the supplement is administered daily from 1 month to 5 months after the procedure. In one embodiment, the nutritional supplement being administered before and after the procedure, and in other embodiments, the nutritional supplement preparation is administered on the day of the procedure. In particular embodiments, the nutritional supplement is administered before and on the day of the procedure, and in other embodiments, the nutritional supplement is administered on the day of the procedure, and after the procedure. In one particular procedure, the nutritional supplement is administered before, the day of, and after, the procedure. The supplement can be administered more than one time per day.

In some embodiments, the non-invasive tissue remodeling procedure performed is suction massage, suction massage and thermal energy, radiofrequency energy, high frequency-focused ultrasound energy, cryolipolysis, or low level light laser energy. In one embodiment, the non-invasive tissue remodeling procedure performed is cryolipolysis.

In another aspect, the disclosure provides a method of providing a nutritional supplement regimen to a subject, comprising administering to the subject one or more dosages of a nutritional supplement, the one or more dosages of the nutritional supplement providing: cholecalciferol at 400 IU to 10,000 IU; genistein at 10 mg to 250 mg; trans-resveratrol at 30 mg to 1,000 mg; and quercetin dihydrate at 40 mg to 1,000 mg.

In some embodiments, the dose of nutritional supplement is administered daily. In certain embodiments, the dose of nutritional supplement is delivered more than once a day. In particular embodiments, the dose of nutritional supplement is administered for from one day to eight months.

In yet another aspect, the disclosure provides a method of preparing the nutritional supplement preparations described above, comprising sieving each of the Vitamin D, genistein, resveratrol, and quercetin ingredients, mixing the component ingredients together, and then encapsulating the mixture in a dose form.

In another aspect, the disclosure provides a nutritional supplement preparation as described above for use as a pharmaceutical composition. Also provided is a nutritional supplement preparation as described above for use in enhancing the outcome of a non-invasive tissue remodeling procedure In another aspect the use of a nutritional supplement preparation described above is provided for enhancing the outcome of a non-invasive tissue remodeling procedure.

### BRIEF DESCRIPTION OF THE FIGURES

The foregoing and other objects of the present disclosure, the various features thereof, as well as the invention itself may be more fully understood from the following description, when read together with the accompanying drawings in which:

FIG. 1A is a baseline time point photographic image taken of a subject taking the supplement and undergoing Coolsculpting of the abdomen (front abdomen position marked as “1”) overlaid with the 4 week follow-up image (front abdomen position marked as “2”);

FIG. 1B is a baseline time point photographic image (front abdomen position marked as “1”) overlaid with the 8 week follow-up image (front abdomen position marked as “3”);

FIG. 1C is a baseline time point photographic image (front abdomen position marked as “1”) overlaid with the 12 week follow-up image (front abdomen position marked as “4”);

FIG. 1D baseline time point photographic image (front abdomen position marked as “1”) overlaid with the 4 week, 8 week, and 12 week follow-up images (front abdomen positions marked as “2-4” respectively), with the measurement line illustrated, wherein the line measures the longitude of the area of treatment at the baseline time point from the back to the most protruding point of the abdomen;

FIG. 2A is a baseline time point photographic image taken of a control group subject undergoing Coolsculpting of the abdomen (front abdomen position marked as “1”) overlaid with the 4 week follow-up image (front abdomen position marked as “2”);

FIG. 2B is a baseline time point photographic image (front abdomen position marked as “1”) overlaid with the 8 week follow-up image (front abdomen position marked as “3”);

FIG. 2C is a overlaid with the 12 week follow-up image (front abdomen position marked as “4”); and

FIG. 2D is a baseline time point photographic image (front abdomen position marked as “1”) overlaid with the 4 week, 8 week, and 12 week follow-up images (front abdomen positions marked as “2-4” respectively), with the measurement line illustrated, wherein the line measures the longitude of the area of treatment at the baseline time point from the back to the most protruding point of the abdomen

### DESCRIPTION

The issued U.S. patents, allowed applications, published foreign applications, and references that are cited herein are hereby incorporated by reference in their entirety to the same extent as if each was specifically and individually indicated to be incorporated by reference. Patent and scientific literature referred to herein establishes knowledge that is available to those of skill in the art.

Unless otherwise defined, all technical and scientific terms used herein have the same meaning as commonly understood by one of ordinary skill in the art to which this disclosure belongs.

It has been discovered that a combination of select concentrations of Vitamin D3, genistein, trans-resveratrol, and quercetin dihydrate synergistically supports the body's natural responses to certain non-invasive tissue remodeling procedures. As such, this supplement is used as part of a method of enhancing a non-invasive tissue remodeling procedure.

### A. Nutritional Supplement Preparations

One nutritional supplement of the present disclosure has four main ingredients: Vitamin D, genistein, resveratrol, and quercetin. These compounds have previously been shown individually to impact the adipogenic development of the mesenchymal stem cells, with simultaneous modulation of multiple molecular targets and effects on different stages of the adipocyte life cycle that ultimately translate in less adipose tissue.

Genistein is a phytoestrogen and belongs to the category of isoflavones. It has been shown to have effects for obesity, diabetes, and cardiovascular diseases, and in vitro, has been shown to inhibit lipid accumulation and decrease the nonesterified fatty acid (NEFA) content of certain mammalian cells. Genistein has also been found to inhibit preadipocyte proliferation, trigger lipolysis, and induces apoptosis in maturing preadipocytes. In combination with Vitamin D, genistein can induce apoptosis and suppress lipid intake in maturing preadipocytes.

Vitamin D is a group of fat-soluble secosteroids responsible for enhancing intestinal absorption of calcium, iron, magnesium, phosphate, and zinc. Cholecalciferol (Vitamin D3) and its precursor, ergocalciferol (Vitamin D2) can be ingested from the diet and from supplements (Baile et al. (2011) Ann. N.Y. Acad. Sci. 1215:40-47; Kim et al. (2006) J. Nutr. 136:409-414; Aguirre et al. (2011) Open Nutraceuticals J4:189-198). The human body can also synthesize cholecalciferol in the skin from cholesterol with sun exposure. There are conflicting reports of the ability of Vitamin D to affect body fat mass and adipogenesis. For example, some studies have demonstrated that Vitamin D metabolites influence adipokine production and the inflammatory response in adipose tissue (Ding et al. (2012) Br. J. Nutr. 108(11):1915-23). Vitamin D has also been shown to have direct (non-calcemic) effects on adipocytes, and to suppress lipid accumulation and increase energy expenditure and fat oxidation. For example, Vitamin D supplementation to women during weight loss did not increase weight loss or associated factors compared with placebo (Mason et al. (2014) Am. J. Clin. Nutr. 99(5):1015-1025). In contrast, Vitamin D3 has been shown to stimulate adipogenesis in mice (Nimitphong et al. (2012) PLOS One 7(12):e52171.doi: 10.1371/journal.pone.0052171.Epub 2012 December 18). Thus, the activity of Vitamin D appears to be variable, if not inconsistent.

Quercetin is a plant-derived, dietary flavonoid with potentially beneficial effects on cardiovascular diseases (Arts et al. (2005) Am. J. Clin. Nutr. 81:317S-325S; Hertog et al. (1993) Lancet, 342:1007-1011). It has been shown to inhibit glucose uptake in isolated rat adipocytes (Strobel et al. (2005) Biochem. J. 386(3):471-478) and to increase lipolysis, an effect that was synergistic with epinephrine (Kuppusamy et al. (1992) Biochem. Pharmacol. 44(7):1307-1315). Quercetin also has been shown to reduce cell proliferation and to cause cell cycle arrest (Yoshida et al. (1992) Canc. Res. 52(23):6676-6681) and apoptosis in vitro (Liesveld et al. (2003) Leukemia Res. 27(6):517-527; Wang et al. (1999) Eur. Canc. J. 35(10):1517-1525; Hsu et al. (2006) Mol. Nutri. Food Res. 50(11):1072-1079). Quercetin inhibits preadipocyte proliferation and lipid accumulation, induces preadipocyte apoptosis, and stimulates lipolysis in mature adipocytes.

Quercetin also aids resveratrol bioavailability and promotes its effects (De Santi et al. (2000) Xenobiotica 30:857-66). In mature adipocytes, the combination of resveratrol and quercetin caused an enhanced increase in apoptosis compared to a predicted additive response, and in maturing preadipocytes, the combination caused an enhanced inhibition of adipogenesis compared to the predicted additive response (Yang et al. (2008) Life Sci. 82:1032-1039). One non-limiting useful form of quercetin is quercetin dihydrate.

Resveratrol (3,5,4′-trihydroxy-trans-stilbene) is produced by mammals in response to injury (Sun et al. (2004) FASEB J. 18:1430-1432). It is a stilbenoid, a type of natural phenol, and a phytoalexin produced naturally by several plants when under attack by pathogens such as bacteria or fungi. Resveratrol has been shown to decrease adipogenesis and osteogenesis promotion, suppress viability in maturing preadipocytes (thereby inhibiting lipid accumulation), and increase osteogenesis and lipolysis in mature adipocytes (Baile et a. supra; Kim et al., supra; Aguirre et al., supra; Kong et al. (2006) Am. J. Physiol. Endocrinol. Metab. 290: E916-E924; Sun et al., supra; Parikh et al. (2004) J. Clin. Endocrinol. Metab. 89:1196-1199; Caron-Jobin et al. (2011) Obesity 19:1335-1341; Lappe et al. (2011) J. Evid. Based Complementary Altern. Med. 16:58-72; Rayalam et al. (2008) Phytother. Res. 22(10):1367-1371; Rayalam et al. (2007) J. Nutr. 137:2668-2673; Yang et al. (2008) Life Sci. 82:1032-1039; Park, et al. (2008) J. Med. Food 11:773-783; Lai, et al. (2011) J. Med. Food 14(11):1352-1362). One useful, non-limiting form of resveratrol is trans-resveratrol.

Resveratrol has been shown to inhibit preadipocyte proliferation and adipogenic differentiation in a Sirt1-dependent manner. In human adipocytes, resveratrol has been demonstrated to stimulate basal- and insulin-stimulated glucose uptake, and to inhibit de novo lipogenesis in parallel with a down-regulation of lipogenic gene expression. (Fischer-Posovszky et al. (2010) Am. J. Clin. Nutr. 292(1):5-15). In 3T3-L1 adipocytes, resveratrol induces apoptosis and inhibits adipogenesis (Rayalam et al., supra).

However, none of these compounds, alone or in combination, have been shown to support the body's natural processes in response to an invasive or non-invasive tissue remodeling procedure, thereby enhancing the aesthetic outcome of such a procedure, and resulting in an improvement in the visible appearance of treated tissues.

The combination of these ingredients in the present nutritional supplement appears to act synergistically to decrease the time it takes for certain initial and maximum results of the non-invasive tissue remodeling procedure to become visible, and to enhance the aesthetic appearance of the treated area. Without being held to any particular mechanism, selected activities of Vitamin D appears to be more consistently available and useful when it is in the presence of genistein, resveratrol and quercetin, and these activities appear to support and increase the body's response to the procedure, whether it is a reduction in adipocyte size, a reduction in its ability to proliferate, or an increase in apoptosis (programmed cell death) or cell death due to injury. In the performance of a body sculpting procedure, certain fat cells and fatty tissues may undergo lethal or sub-lethal damage. The synergistic support of the present nutritional supplements work by both sensitizing the fat cells to the procedure damage and permitting the sub-lethal fat cell damage to become lethal, thereby improving procedure effectiveness. The nutritional supplements also enhance biological pathways in the body responsible for clearance of such released fat from the damaged adipose cells, leading to a more rapid observable procedure effect.

All of these component ingredients can be extracted from foods or can be commercially obtained. The supplement may contain other non-active ingredients as described below. All of these additional other ingredients are commercially available as well.

Effective amounts of the four main ingredients have been established by one skilled in the art. However, it is within the skilled artisan's purview to determine other optimal effective amount ranges for a subject undergoing a tissue remodeling procedure. The amount of the main ingredients which can be combined with a carrier material to produce a single dosage form will vary depending upon the subject undergoing the tissue remodeling procedure. The amount of each of the four main ingredients, which can be combined with a carrier material to produce a single dosage form, will generally be that amount of the combined ingredients which produce a supportive effect.

One non-limiting, representative nutritional supplement comprises Vitamin D3 (cholecalciferol), genistein, trans-resveratrol, and quercetin dihydrate. For example, effective amounts of Vitamin D3 (cholecalciferol) are from about 400 IU to about 10,000 IU, from about 600 IU to about 5,000 IU, from about 1,000 IU to about 5,000 IU, or about 1,000 IU, about 2,000 IU, about 3,000 IU, about 4,000 IU or about 5,000 is provided as the serving and/or daily dose. Trans-resveratrol is present in the supplement at from about 30 mg to about 1,000 mg, from about 50 mg to about 600 mg, from about 100 mg to about 500 mg, about 100 mg, about 150 mg, about 200 mg, about 250 mg, about 300 mg, about 350 mg, about 400 mg, or about 500 mg as the serving and/or daily dose. Quercetin is present in dihydrate form in the supplement at from about 40 mg to about 1,000 mg, from about 100 mg to about 800 mg, from about 100 mg to about 500 mg, or about 100 mg, about 200 mg, about 300 mg, about 400 mg, or about 500 mg as the serving and/or daily dose. Genistein is present in the supplement at from about 10 mg to about 250 mg, from about 20 mg to about 125 mg, from about 17 mg to about 100 mg, or about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg as the serving and/or daily dose.

The four ingredients can be combined with a pharmaceutically-acceptable excipient, carrier, or diluent, depending on the form that the supplement takes. The phrase “pharmaceutically-acceptable excipient, carrier, or diluent” as used herein means a pharmaceutically-acceptable material, composition or vehicle, such as a liquid or solid filler, diluent, excipient, solvent, or encapsulating material, involved in carrying or transporting the main ingredients from one portion of the body to another part of the body. Each carrier must be “acceptable” in the sense of being compatible with the other ingredients of the formulation and not injurious to the subject. Some examples of materials which can serve as pharmaceutically-acceptable carriers include: sugars, such as food glaze, lactose, glucose, and sucrose; starches, such as maltodextrin, corn starch, rice starch, and potato starch; cellulose, and its derivatives, such as sodium carboxymethyl cellulose, ethyl cellulose, and cellulose acetate; powdered tragacanth; malt; gelatin; talc; excipients, such as silica dioxide cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers and lubricants, such as sodium lauryl sulfate, magnesium stearate, and polyethylene oxide-polypropylene oxide copolymer as well as coloring agents, release agents, coating agents, sweetening, flavoring, and perfuming agents, preservatives and antioxidants can also be present in the compositions.

The nutritional supplement can be in a form suitable for oral administration. The formulations may conveniently be presented in unit dosage form and may be prepared by any methods well known in the art of pharmacy.

Nutritional supplements according to the disclosure suitable for oral administration may be in the form of capsules, cachets, pills, tablets, lozenges (using, e.g., a flavored basis, such as a sweetener), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), each containing a predetermined amount of the four main ingredients of the present nutritional supplement.

In solid dosage forms for oral administration (capsules, tablets, pills, dragees, powders, granules, and the like), the four main ingredients are mixed with one or more pharmaceutically-acceptable carriers and/or any of the following: fillers, extenders, or excipients, such as starches, lactose, sucrose, glucose, mannitol, and/or silicic acid, and/or high molecular weight polyethylene glycols, and the like; binders, such as, for example, carboxymethylcellulose, alginates, gelatin, polyvinyl pyrrolidone, sucrose, and/or acacia; humectants, such as glycerol; disintegrating agents, such as agar-agar, calcium carbonate, potato or tapioca starch, alginic acid, certain silicates, sodium carbonate, and sodium starch glycolate; solution retarding agents, such as paraffin; absorption accelerators, such as quaternary ammonium compounds; wetting agents, such as, for example, cetyl alcohol, glycerol monostearate, and polyethylene oxide-polypropylene oxide copolymer; absorbents, such as kaolin and bentonite clay; coloring agents; and buffering agents.

Liquid dosage forms for oral administration of the compounds of the disclosure include pharmaceutically acceptable emulsions, microemulsions, solutions, suspensions, syrups, and elixirs. In addition to the four main ingredients, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols, and fatty acid esters of sorbitan, and mixtures thereof. Additionally, cyclodextrins, e.g., hydroxypropyl-beta-cyclodextrin, may be used to solubilize compounds.

Powders can contain, in addition to the four main ingredients, excipients, such as lactose, talc, silicic acid, aluminum hydroxide, calcium silicates, and polyamide powder, or mixtures of these substances.

In some cases, in order to prolong the effect of the nutritional supplement according to the disclosure, it is useful to slow the absorption of the main ingredients. This may be accomplished by using a thick or multiple pill coating(s) or by using very slowly dissolving capsule. The rate of absorption of the four main ingredients then depends upon the rate of dissolution of the coating or capsule.

Those with skill in the art are aware of methods of preparing pills, capsules, and the like. For example, to prepare the nutritional supplement in the form of a capsule, the initial manufacturing process entails dispensing or weighing the appropriate amounts of each of the ingredients, blending or mixing of the ingredients, and encapsulating the blended ingredients into empty capsule shells or forming it into pills, which are then coated, or into powders or crystals that can be dissolved or mixed in an aqueous solution.

### B. Non-Invasive Tissue Remodeling Procedures

The non-invasive tissue remodeling procedures which can be enhanced by the nutritional supplement according to the disclosure are many. These procedures tend to be classified on the basis of the type of energy delivered by a particular technology in modifying the adipocyte. Some of these procedures are shown below in Table 1 (Mulholland et al., supra).

TABLE 1

| Classification | Exemplary Procedure |
|---|---|
| Suction: Massage | Endermologie, France |
| Devices | |
| Suction-Massage: | TriActive (Cynosure, Inc., Westford, MA); |
| Thermal Devices | Smoothshapes (Cynosure, Inc., Westford, MA) |
| Radiofrequency Energy | VelaSmooth, VelaShape (Syneron, Inc., |
| Devices | Irvine, CA); |
| | Thermage# (Solta Medical, Hayward, CA); |
| | Accent (Alma Lasers Inc., Buffalo Grove, IL); |
| | TiteFX (Invasix, Inc., Yokneam, Israel) |
| High-Frequency Focused | UltraShape (UltraShape Ltd., Yoqneam, Israel); |
| Ultrasound Energy | LipoSonix (Medicis, Scottsdale, AZ) |
| Devices | |
| Cryolipolysis (Cold) | Zeltiq (Zeltiq Aaesthetics, Pleasanton, CA) |
| Energy Devices | |
| Low-Level Light | Zerona (Erchonia Medical, McKinney, TX) |
| Laser Therapy Devices | |

Most of these thermal technologies do not kill adipocyte or other surrounding cells in the fatty tissue. Some technologies deploy energy, either a pulse of high-voltage RF current, or a focused high-frequency ultrasound energy experience that disables or destroys the adipocyte by permanently damaging the cell membrane, or coagulating or disrupting and releasing the adipocyte cell contents such as low-level light laser therapy, create temporary disruptions in the cell membrane of the adipocyte allowing a temporary egress of the triglyceride from the cytoplasm, but the cell membrane then corrects itself. Accordingly, through the mechanisms of thermal augmentation of normal metabolic pathways, thermal destruction, cavitational destruction and/or an energy cascade and creation of a temporary adipocyte cell membrane pore, the final result is a temporary or permanent size reduction of the adipocytes and/or the number of adipocytes are reduced, which when translated over hundreds of thousands or millions of fat cells, can result in a measurable reduction of fat and a circumferential reduction of the body contour area in the treated area. The compositions according to the present disclosure can work to assist with any/all of these mechanisms, and thus with many different non-invasive body sculpting procedures involving thermal technologies.

### C. Cryolipolysis

One particular non-invasive tissue remodeling procedure that can be enhanced by the nutritional supplement according to the disclosure is cryolipolysis. A targeted area of a subject's body is drawn up with mild suction and the tissue is held between the panels of the treatment cup or the targeted area is pressed against a flat applicator consisting of cold panels for 30 to 120 minutes during which they apply cooling temperatures to the skin in a range of from about −20° C. to about 20° C. (see, e.g., U.S. Pub. No. 2010/0280582). The amount of cooling (selected energy extraction rate) is controlled by thermoelectric cooling cells powered by DC current and controlled by thermistors that monitor the skin temperature. Up to 20% of patients experience mild transient reduction in sensation (dysesthesia) that returned spontaneously with 7 weeks of treatment, but there are no reports of permanent sensory loss (Mulholland et al., supra).

When cooling subcutaneous tissues to a temperature lower than 37° C., subcutaneous lipid-rich cells, such as adipocytes, can be selectively affected. In general, the epidermis and dermis lack lipid-rich cells compared to the underlying lipid-rich cells forming the adipose tissue. Because non-lipid-rich cells usually can withstand colder temperatures better than lipid-rich cells, the subcutaneous lipid-rich cells can be affected selectively without affecting the non-lipid-rich cells in the dermis, epidermis, and other surrounding tissue (see, e.g., U.S. Pub. No. 2010/0280582).

Without being bound by theory, the selective effect of cooling on lipid-rich cells is believed to result in, for example, membrane disruption, cell shrinkage, or disabling, destroying, removing, killing, or other method of altering lipid-rich cells. Cold exposure may also cause the onset of an inflammatory reaction within the treated adipose tissue. The mechanism for this phenomenon may be a cold-induced apoptotic adipocyte cell death for those fat cells that have been exposed to a cold stimulus that is above freezing but below body temperatures for a defined duration. Another mechanism of apoptotic lipid-rich cell death by cooling is believed to involve localized crystallization of lipids within the adipocytes at temperatures that do not induce crystallization in non-lipid-rich cells. The crystallized lipids selectively may injure these cells, inducing apoptosis (and may also induce necrotic death if the crystallized lipids damage or rupture the bi-lipid membrane of the adipocyte) (see, e.g., Mulholland et al., supra). Yet other mechanisms of injury may involve the lipid phase transition of those lipids within the cell's bi-lipid membrane, which results in membrane disruption, thereby compromising the integrity and/or function of the cell membrane and inducing apoptosis (see, e.g., Quinn (1985) Cryobiol. 22:128-147; Rubinsky (2003) Heart Failure Rev., 8:277-284). Local cold exposure also is believed to induce lipolysis (i.e., fat metabolism) of lipid-rich cells, and has been shown to enhance existing lipolysis which serves to further increase the reduction in subcutaneous lipid-rich cells (Vallerand et al., (1999) Aviation, Space Environ. Med. 70:42-50). Cold sensitivity of lipid-rich cells may be variable under certain circumstances and can lead to a certain variability of the threshold temperature needed to trigger the apoptotic or cell death event that is needed to lead to subcutaneous reduction of fat cells.

### D. Methodologies

An amount of the nutritional supplement preparation according to the disclosure is provided to the subject in one method according to the disclosure. For example, the preparation is provided for a certain period of time before undergoing a non-invasive tissue remodeling procedure. The subject also or alternatively takes that amount of the supplement for a certain period of time after the procedure. The preparation may also be provided on the day of the procedure.

In another method of the present disclosure, a supplement comprising Vitamin D3 (or Vitamin D3 and at least one of trans-resveratrol, quercetin dihydrate, or genistein) is provided (instead of the nutritional supplement preparation comprising all four of these ingredients) in a carrier is administered a certain period of time before and/or after undergoing a non-invasive tissue remodeling procedure. This supplement may also be provided the day of the procedure.

The amount of the nutritional supplement in the oral unit dosage form, with as a single or multiple dosage, is an amount that is effective for supporting a tissue remodeling procedure. The precise dose to be taken by the subject as directed by the physician performing the tissue remodeling procedure will depend on a variety of factors, examples of which include the specific type of tissue remodeling procedure being performed, as well as various physical factors related to the individual undergoing the procedure, such as their health and their level of bioavailable Vitamin D.

For example, the nutritional supplement preparation taken from one to about 60 days before the procedure, from 7 days to about 30 days before the procedure, from 5 to about 50 days before the procedure, from 4 to about 20 days before the procedure from one to about 14 days before the procedure, or 1 day, 2 days, 3 days, 4 days, 5 days, 6 days, 7 days, 8 days, 9 days, 10 days, 11 days, 12 days, 13 days, or 14 days before the procedure. The nutritional supplement preparation may also be taken from about 1 month to about 5 months after the procedure, from about 2 months to about 4 months after the procedure, from about 3 months to about 5 months after the procedure, or for about 1 month, about 2 months, about 3 months, about 4 months, or about 5 months after the procedure. The nutritional supplement preparation may also be taken on the day of the procedure.

In one method of the present disclosure, the body area of the patient who will be undergoing the tissue remodeling procedure is examined before and after the procedure. This body area is any part of the patient's body for which an improved aesthetic appearance is desired. For example, the targeted body area can be the hips, “love-handles,” abdomen, buttocks, arms, neck, flanks, and/or back. An improved aesthetic appearance of the targeted area may include a more consistent, even-textured, and smooth skin surface, and/or a decrease in the size of the targeted body area.

Examination can be performed by visual inspection of the body area which can be memorialized and further examined and analyzed via photographic images. Measurements of the tissue in the body area can be taken, e.g., by image analysis via calipers or other device known to those with skill in the art. The weight of the patient and other physiological measurements may also be taken. This examination can be performed multiple times of increasing length (e.g., days, weeks, months) after the procedure to measure the progress of the outcome of the procedure. It has been determined by such examination that administration of the nutritional supplement according to the disclosure results in an enhanced body response to the procedure (e.g., a shorter time to detect an improved aesthetic appearance of the area).

Reference will now be made to specific examples illustrating the disclosure. It is to be understood that the examples are provided to illustrate exemplary embodiments and that no limitation to the scope of the disclosure is intended thereby.

### EXAMPLES

### Example 1

### Preparation of an Encapsulated Form of the Nutritional Supplement

The four component ingredients (Vitamin D3, genistein, trans-resveratrol, and quercetin dihydrate) as well as the other secondary ingredients listed were obtained from the commercial sources listed below.

| | | City, State |
|---|---|---|
| | | (or City, Country |
| Ingredients | Manufacture | if outside US) |
| Vitamin D3 850 CWS Powder | Supreem Pharmaceuticals Mysore Pvt. Ltd. | Geneva, |
| | | Switzerland |
| Silicon Dioxide (GL 200) | Glassven-Yangzhong Silicas and | Yangzhong City, |
| | Chemicals JV Ltd. | China |
| Dicalcium Phosphate | Jiangsu Chengxing Phosph-Chemicals Co. | Jiangsu, China |
| Dihydrate | Ltd. | |
| Magnesium Stearate | Faci Asia Pacific Pte. Ltd. | Singapore |
| Empty Hard Capsules of | CapsCanada | Pompano Beach, |
| Vegetable Origin Size “0,” | | FL |
| Natural | | |
| Genistein (GeniVida TG) | DSM Nutritional Products Inc. | Parsippany-Troy |
| | | Hills, NJ |
| Resveratrol 99% | Pharma Science Nutrients Inc. | Dunedin, FL |
| Quercetin PIE 95% | Glory Food Inc. | Walnut, CA |

Each of the component ingredients are tested for purity and released by Quality Control into the dispensing and blending area. The component ingredients are inspected (visually), weighed on calibrated scales (Precision scale-DWP-102 E) and placed in polyethylene bags for blending and mixing purposes. At this stage, each of the component materials are sieved (mesh size 10-40) to remove lumps or agglomerates. The components are then blended using a clean V-Blender (Patterson-Kelly cross flow blender 2 cubic ft) for at least 10 min. A lubricant (magnesium stearate) is then added and blending is carried out for a further 4 min. The blended material is collected in polyethylene bags and placed in drums and transferred to an encapsulation room. The equipment used for encapsulation is a semi-automatic CGN 208-D Capsule Filling Machine. The machine consists of two detachable pattern plates (one for the empty capsules shells and another for fitting caps onto the filled capsule bodies) and a hopper for filling the raw material blend. Once the capsule is filled with the blend, the two plates are brought together and through an in-built mechanism, the caps are fitted onto the bodies of the capsule. The finished capsules are then placed in the designated containers and desiccants may be added if required to these containers.

### Example 2

### Validation Study

A validation study was performed to demonstrate that the nutritional supplement preparations according to the disclosure enhances the efficacy of a non-invasive tissue remodeling procedure (e.g., cryolyposis or “CoolScuplting™” (“CS”)) performed on a subject. A batch of nutritional supplement was prepared to validate that the supplement provides the desired increase or enhancement of the efficacy and outcome of its companion procedure.

The validation study was conducted under the supervision of a physician. All enrolled participants were screened for eligibility via (1) a health questionnaire that focuses on their current dietary intake and general health including any current medications or supplements being taken, and (2) physical examination. Subjects taking the nutritional supplement preparation were not on Vitamin D supplementation therapy, nor were known to have Vitamin D deficiency (whether on current Vitamin D replacement or not), and did not have a history of parathyroid disease.

The study included 50 voluntary participants, half of whom received the nutritional supplement preparation described above in Example 1, and the other half received nothing, thus acting as a (negative) control group.

Participants in the study taking the supplement receive instructions on the usage of the supplement as follows: take two capsules, twice daily with meals for a period of 8 wk after the tissue remodeling procedure.

All subjects underwent between one and eight CS treatment cycles at areas such as: the abdomen, love handles, back, flanks, arms, or thighs.

Efficacy validation was determined, in part, by comparison of “before and after” measurements, including weight, caliper measurements of fat or skin folds (see, e.g., Hu, (ed.) Obesity Epidemiol. (New York City: Oxford University Press), (2008) pp. 53-83), and appearance of treated areas. For example, caliper measurements and other “before and after” measurements were taken at consultation and prior to the procedure (which may also be 2 wk after beginning pretreatment); and at each follow up visit (e.g., at 4 wk, 8 wk, and 12 wk) post-CS procedure.

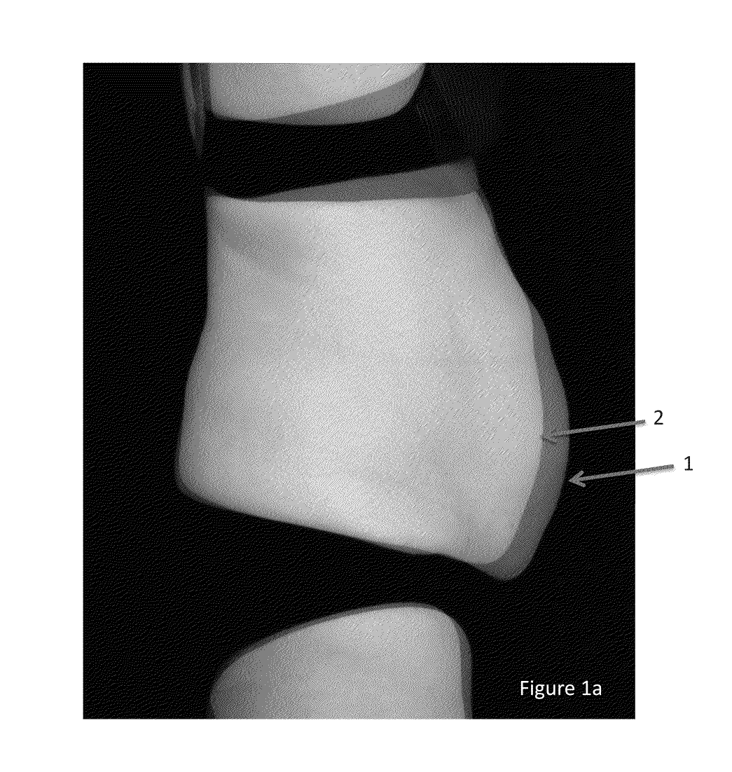

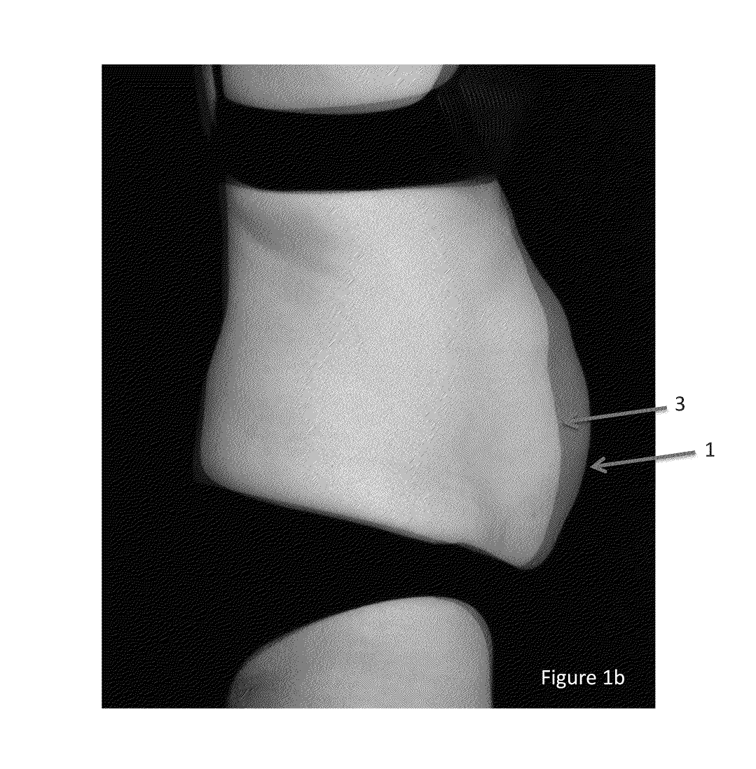

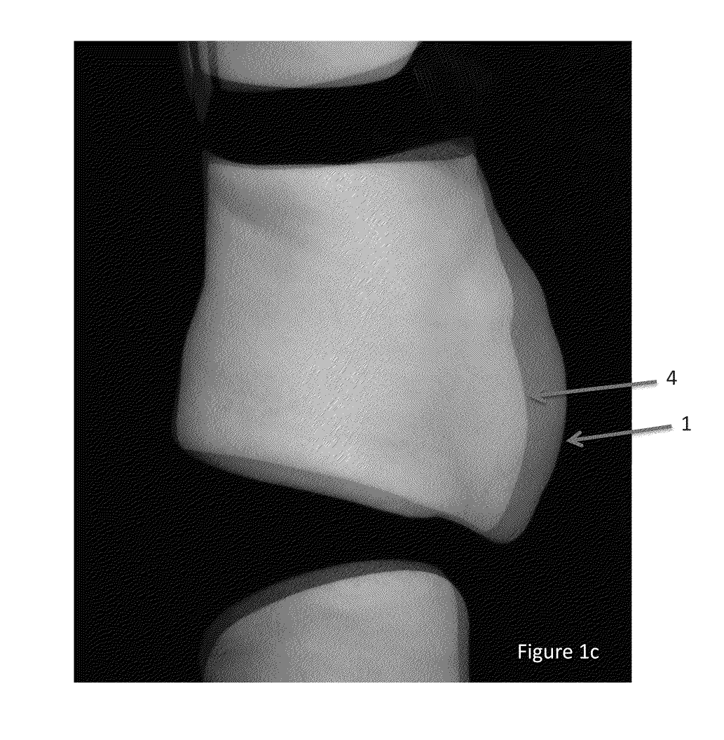

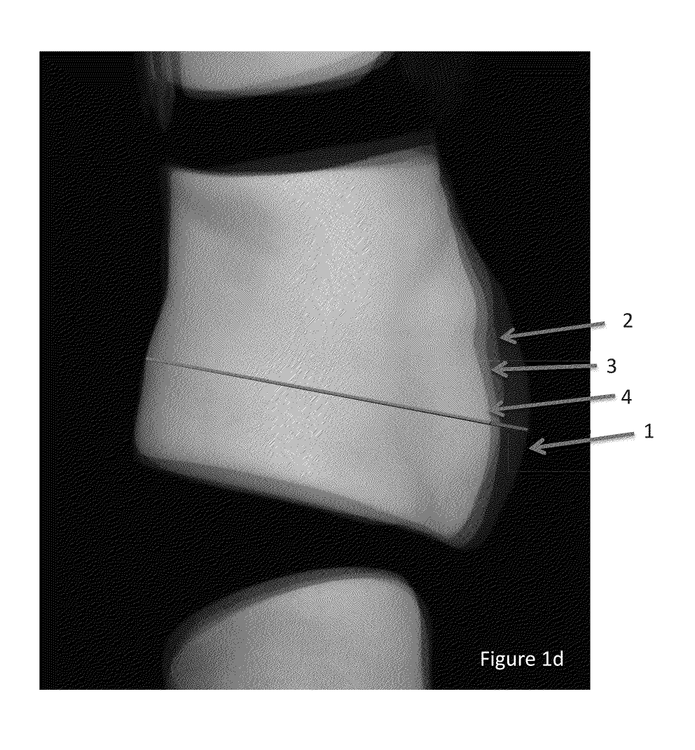

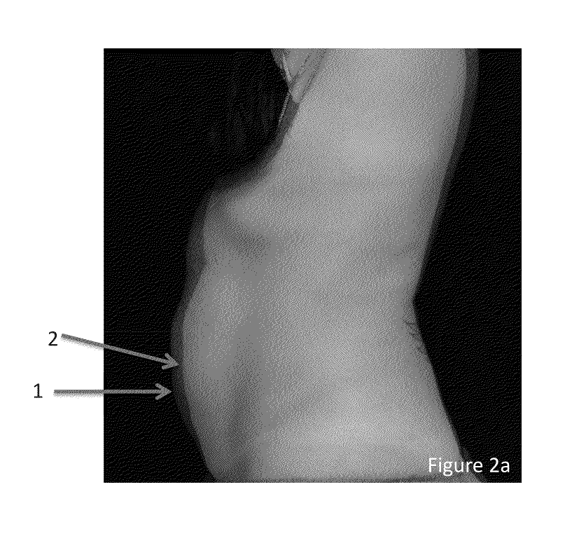

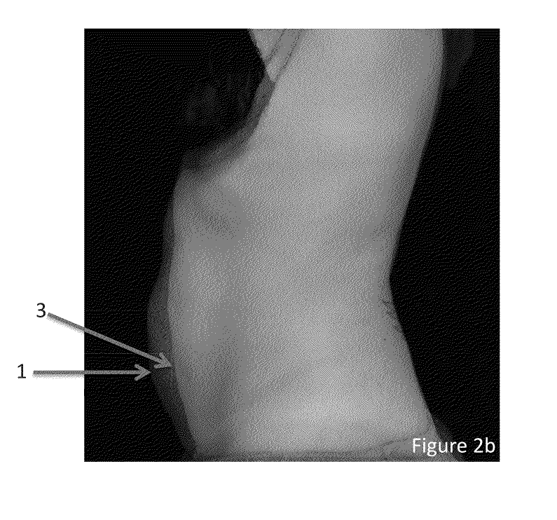

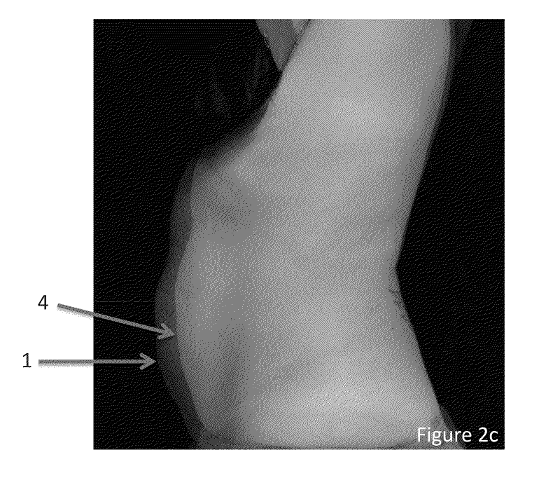

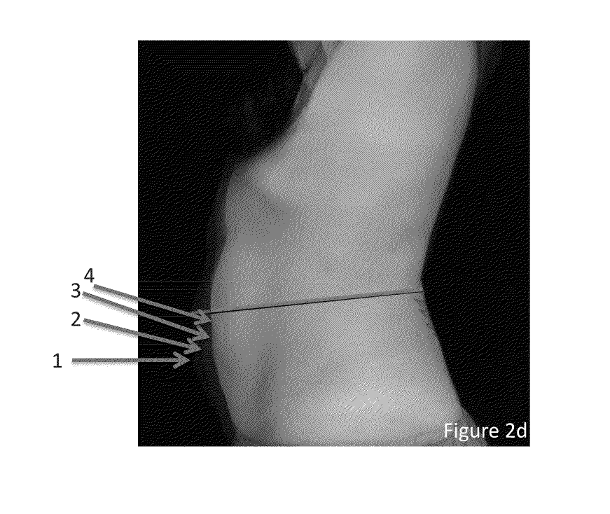

Photographs of the treated areas were taken once before (i.e., the baseline), and then at 4 wk, 8 wk, and 12 wk after, the procedure (see, FIGS. 1A-1C (+supplement), FIGS. 2A-2C (no supplement). The photographs were standardized pictures taken with consistent photography, lighting, and background conditions. During the photography sessions, each subject (patient) wears identical, black undergarments (i.e., sports bra and panties for females, briefs for males) to further standardize the photography process. The FotoFinder photography (Camera: CANON EOS Rebel T3i, Lens: EF-S 18-55 mm 1:3.5-5.6 IS II, Lens Zoom settings for body pictures: 35 mm) and software (Version 119) system (FOTOFINDER SYSTEMS, Inc., Bad Birbaum, Germany) was useful in this regard. The Zeltiq floor octagon was used for standardized rotation of body photography. The camera settings are as follows:

| Light Skin | Medium Skin | Dark Skin |
|---|---|---|
| Av: 25 | Av: 22 | Av: 20 |
| Tv: 1/125 | Tv: 1/125 | Tv: 1/125 |
| ISO: 100 | ISO: 100 | ISO: 100 |

The Fotofinder Laser-liner was used for aligning subjects (patients) to a given distance of 90 centimeters (cm). This is a battery-operated Laser-Product that displays a continuous red line on the floor. A black background was used on all of the pictures. The FotoFinder system provides a “ghost” image that mimics the exact position of the subject during photography at the baseline time point when taking follow-up photographs to further insure consistency in measurement.

The photographs were further analyzed using the Adobe Photoshop program to determine “before and after” measurements, in millimeters, of the treated body areas. By using the millimeter scale ruler embedded in the program, a line was drawn between two consistent points on the photograph taken of each subject for the baseline time point; thus giving a baseline measurement by which post-CS photographic measurements were compared. Photographic overlays of the 4 wk, 8 wk, and 12 wk post-CS time points were created in the program by reducing the opacity of the post-CS images by 50% and superimposing each post-CS time point (i.e. 4 wk, 8 wk, and 12 wk) photograph over the baseline image. The difference in millimeters of the position on the line of the treated body part with respect to the baseline measurement at each post-CS time point was noted. For the purposes of illustration, examples of such overlays and measurements are shown in FIGS. 1A-1C (+supplement) and FIGS. 2A-2C (no supplement). The results are summarized in FIG. 1D and FIG. 2D.

Administering the nutritional supplement preparation in the amounts and at the times described above resulted in a noticeable reduction in size of the area(s) treated during the CS procedure in as little as 4 wk post-treatment (see, FIG. 1A vs. FIG. 2A (4 wk); FIG. 1B vs. FIG. 2B (8 wk); FIG. 1C vs. FIG. 2C (12 wk)), as well as, a greater reduction of fatty tissue in subjects receiving the nutritional supplement. At any given observed post-CS time point (4 wk, 8 wk, and 12 wk), the reduction in the total number of mm of fat accumulation was consistently higher (7.9 mm, 6.5 mm, and 6.9 mm higher, with an average of 7.1 mm) in the group of subjects treated with the preparation, with respect to the control group. An analogous pattern was found when considering the percentages of total reduction of mm of fat accumulation: the group of patients that received the preparation showed a reduction of total fat accumulation that is 1.1%, 0.6%, and 0.2% higher, than in the control group, observed at 4 wk, 8 wk, and 12 wk post-CS, respectively. Subjects not receiving the nutritional supplement exhibit results typical of patients undergoing a traditional CS procedure; reduction in size is noticeable on average at 8 wk to 12 wk post-procedure, and to a lesser amount on average when compared to subjects receiving the nutritional supplement.

### EQUIVALENTS

Those skilled in the art will recognize, or be able to ascertain, using no more than routine experimentation, numerous equivalents to the specific composition and procedures described herein. Such equivalents are considered to be within the scope of this disclosure, and are covered by the following claims.

1. A nutritional supplement preparation comprising:
Vitamin D at 400 IU to 10,000 IU;
genistein at 10 mg to 250 mg;
resveratrol at 30 mg to 1,000 mg; and
quercetin at 40 mg to 1,000 mg.

2. The nutritional supplement preparation of claim 1, wherein Vitamin D is Vitamin D2, Vitamin D3 (cholecalciferol), or a combination thereof.

3. The nutritional supplement preparation of claim 1, wherein resveratrol comprises trans-resveratrol.

4. The nutritional supplement preparation of claim 1, wherein quercetin comprises quercetin dihydrate.

5. The nutritional supplement preparation of claim 1, comprising:
cholecalciferol at 400 IU to 10,000 IU;
genistein at 10 mg to 250 mg;
trans-resveratrol at 30 mg to 1,000 mg; and
quercetin dihydrate at 40 mg to 1,000 mg.

6. The nutritional supplement preparation of claim 1, which is in a unit dose form.

7. The supplement preparation of claim 5, wherein:
cholecalciferol is present at 600 IU to 5,000 IU;
genistein is present at 20 mg to 125 mg;
trans-resveratrol is present at 50 mg to 600 mg; and
quercetin dihydrate is present at 100 mg to about 500 mg.

8. The supplement preparation of claim 5, wherein:
cholecalciferol is present at 1,000 IU to 5,000 IU;
genistein is present at 17 mg to 100 mg;
trans-resveratrol is present at 100 mg to 500 mg; and
quercetin dihydrate is present at 100 mg to 200 mg.

9. The supplement preparation of claim 1, further comprising a pharmaceutically-acceptable excipient, carrier, or diluent.

10. The supplement preparation of claim 1, further comprising: a filler; extender; excipient;
binder; humectant; disintegrating agent; solution-retarding agent; wetting agent; absorbent;
coloring agent; buffering agent; and/or combinations thereof.

11. The supplement preparation of claim 1, which is in an orally available form.

12. The supplement preparation of claim 11 in capsule, pill, tablet, dragee, powder, gummy chew, or granular form.

13. The supplement preparation of claim 11 in emulsion, microemulsion, solution, suspension, syrup, or elixir form.

14. The supplement preparation of claim 12, which is in time-release form.

15. A method of enhancing the outcome of a non-invasive tissue remodeling procedure, the outcome comprising an improved aesthetic appearance of a body area having undergone the procedure, the method comprising:
examining the body area of the patient before the procedure;
administering to the subject an amount of a nutritional supplement which supports the body's response to the procedure, the amount of nutritional supplement comprising:
Vitamin D at 400 IU to 10,000 IU;
genistein at 10 mg to 250 mg;
resveratrol at 30 mg to 1,000 mg; and
quercetin at 40 mg to 1,000 mg; and
examining the body area after the procedure has been performed,
wherein the treated body area has an improved aesthetic appearance relative to the appearance of the area before undergoing the procedure.

16. The method of claim 15, wherein the step of examining the body area of a patient that has undergone the procedure is performed multiple times of increasing length after the procedure, and wherein the improved aesthetic appearance of the treated body area is detected in a shorter period time after the procedure relative to the period of time it takes to detect an improved aesthetic appearance of a body area of a patient who has undergone the procedure and who has not been administered the nutritional supplement.

17. The method of claim 16, wherein the examining steps comprise photographing the body area and/or taking caliper measurements of the area.

18. The method of claim 17, further comprising weighing the patient before and after having undergone the treatment.

19. The method of claim 15, wherein the nutritional supplement administered comprises Vitamin D2, Vitamin D3, or a combination thereof.

20. The method of claim 15, wherein the nutritional supplement administered comprises quercetin dihydrate.

21. The method of claim 15, wherein the nutritional supplement administered comprises trans-resveratrol.

22. The method of claim 15, wherein the nutritional supplement administered comprises:
Vitamin D3 at 400 IU to 10,000 IU;
genistein at 10 mg to 250 mg;
trans-resveratrol at 30 mg to 1,000 mg; and
quercetin dihydrate at 40 mg to 1,000 mg,

23. The method of claim 15, wherein the nutritional supplement is administered before the procedure.

24. The method of claim 23, wherein the supplement is administered daily from 1 day to 60 days before the procedure.

25. The method of claim 15, wherein the nutritional supplement being administered after the procedure.

26. The method of claim 25, wherein the supplement is administered daily from 1 month to 5 months after the procedure.

27. The method of claim 15, wherein the nutritional supplement being administered before and after the procedure.

28. The method of claim 15, wherein the nutritional supplement preparation is administered on the day of the procedure.

29. The method of claim 23, further comprising administering the nutritional supplement on the day of the procedure.

30. The method of claim 25, further comprising administering the nutritional supplement on the day of the procedure.

31. The method of claim 15, wherein the supplement is administered more than one time per day.

32. The method of claim 15, wherein the non-invasive tissue remodeling procedure is suction massage, suction massage and thermal energy, radiofrequency energy, high frequency-focused ultrasound energy, cryolipolysis, or low level light laser energy.

33. The method of claim 32, wherein the non-invasive tissue remodeling procedure is cryolipolysis.

34. A method of providing a nutritional supplement regimen to a subject, comprising administering to the subject one or more dosages of a nutritional supplement,
the one or more dosages of the nutritional supplement providing:
cholecalciferol at 400 IU to 10,000 IU;
genistein at 10 mg to 250 mg;
trans-resveratrol at 30 mg to 1,000 mg; and
quercetin dihydrate at 40 mg to 1,000 mg.

35. The method of claim 34, wherein the dose of nutritional supplement is administered daily.

36. The method of claim 34, wherein the dose of nutritional supplement is delivered more than once a day.

37. The method of claim 34, wherein the dose of nutritional supplement is administered for from one day to eight months.

38. A method of preparing the nutritional supplement preparation of claim 1, comprising:
sieving the Vitamin D, genistein, resveratrol, and quercetin ingredients;
mixing the ingredients together; and
encapsulating the mixture in a dose form.
